(19) United States
(12) Patent Application Publication
(10) Pub. No.: US 2006/0286604 A1
(43) Pub. Date: Dec. 21, 2006

(54) Lectin binding to choroidal neovascularization

(75) Inventor: Robert F. Mullins, Coralville, IA (US)

Correspondence Address: FULBRIGHT & JAWORSKI L.L.P., 600 CONGRESS AVE., SUITE 2400, AUSTIN, TX, 78701, US

(73) Assignee: The University of Iowa Research Foundation

(21) Appl. No.: 11/399,299

(22) Filed: Apr. 6, 2006

Related U.S. Application Data
(60) Provisional application No. 60/669,137, filed on Apr. 7, 2005.

(51) Int. Cl. G01N 33/53; A61K 38/16

(52) U.S. Cl. 435/7.1; 514/8

(57) ABSTRACT

The present invention involves the identification of choroidal neovascularization (CNV) based on lectin binding patterns in choroidal and/or Bruch's membranes. The use of lectins to target therapeutic agents to CNV also is disclosed.

The present application claims benefit of priority to U.S. Provisional Application Ser. No. 60/669,137, filed Apr. 7, 2005, the entire contents of which are hereby incorporated by reference.

The government owns rights in the present invention by virtue of funding from NEI (grant no. EY014563).

### BACKGROUND OF THE INVENTION

I. Field of the Invention

The present invention relates generally to the fields of opthamology, pathology and biochemistry. More particularly, it concerns the identification of carbohydrate groups on choroidal neovascular membranes associated with macular degeneration.

II. Description of Related Art

Age-related macular degeneration (AMD) is the most common cause of irreversible vision loss in the developed world (Tielsch et al., 1995; Klayer et al., 1998; Attebo et al., 1996). In some cases, macular degeneration may be active and then slow down considerably, or even stop progressing for many, many years. There are ways to arrest macular degeneration, depending on the type and the degree of the condition. These range from nutritional intervention to laser surgery of the blood vessels. However, a cure has not yet been found, and as a result, AMD is the most common cause of severe visual loss in the developed world, impairing more than 10 million people in the United States alone (Friedman et al., 2004), with approximately 1 in 3 people over the age of 75 are affected to some degree (Klein et al., 1992).

There are two forms of AMD—wet and dry. Both of these forms can be characterized by lesions that lie beneath the retinal pigment epithelium (RPE) and within a multi-layered structure known as Bruch's membrane. The central layer of Bruch's membrane is composed largely of elastin, and this layer is sandwiched between two collagenous sheets. The basal laminae of the RPE (on the retinal side) and the choriocapillaris (on the choroidal side) lie upon these sheets of collagen to complete the five layered structure.

AMD is likely to be a mechanistically heterogeneous group of disorders. At this time, the specific disease mechanisms that underlie the vast majority of cases of age related macular degeneration are unknown. However, a number of studies have suggested that both genetic and environmental factors are likely to play a role in most patients (Heiba et al., 1994; Seddon et al., 1997; Klayer et al., 1998). Several investigators have used a population-based epidemiologic approach to try to identify specific environmental insults that might increase an individual's risk for AMD (Smith et al., 2001; Seddon et al., 1994). These studies have revealed some factors that appear to modify or exacerbate the disease (smoking is the most significant of the latter) (Smith et al., 2001), but none that are likely to be causative. This is perhaps understandable given the high prevalence, late onset, and slow progression of the disease.

Early detection is important because a patient destined to develop macular degeneration can sometimes be treated before symptoms appear, and this may delay or reduce the severity of the disease. Furthermore, as better treatments for macular degeneration are developed, whether medicinal, surgical, or low vision aids, patients diagnosed with macular degeneration can sooner benefit from them. Thus, the identification of distinct markers that differentially label components of the structures involved in AMD could not only yield important insights into the pathogenesis of this condition, but permit early diagnosis. A means of specifically labeling neovascular endothelial cells (EC), as compared with healthy endothelial cells outside of the CNVM, would assist in conventional treatments of AMD. Further, the resulting clinical benefit of a molecularly targeted treatment of AMD would be significant, especially if the abnormal endothelial cells present in CNVMs can be specifically targeted by exploiting molecular differences between neovascular and normal endothelial cells.

### SUMMARY OF THE INVENTION

Thus, in accordance with the present invention, there is provided a method of identifying choroidal neovascularization (CNV) in a subject comprising (a) contacting a choroidal membrane and or a Bruch's membrane of the subject with a lectin; (b) assessing the binding of the lectin to the choroidal membrane and/or Bruch's membrane; and (c) comparing binding patterns of the lectins to the known structure of CNV. Step (a) may comprise contacting a choroidal membrane with the lectin, or contacting a Bruch's membrane with the lectin. Also useful are any other carbohydrate-binding agents, including non-protein molecules, that may be functional substitutes of lectins.

The lectin may be labeled, or may be unlabeled, wherein the method further comprises an additional step of contacting the choroidal membrane or Bruch's membrane with an agent that permits detection of bound lectin. The lectin may be selected from the group consisting of SBA, VVA, UEA-1 and sWGA. The method may further comprise making a treatment decision based on the distribution of lectin binding on said choroidal membrane and/or Bruch's membrane. Steps (a)-(c) may be performed a second time and the results from both identifications are compared.

In another embodiment, there is provided a method of diagnosing wet macular degeneration comprising (a) contacting a choroidal membrane and or a Bruch's membrane of a subject with a lectin; (b) assessing the binding of the lectin to said choroidal membrane and/or Bruch's membrane; and (c) comparing binding patterns of the lectins to the known structure of choroidal neovascularlization (CNV), wherein the identification of a CNV structure in the choroidal membrane or Bruch's membrane is diagnostic of wet macular degeneration. Step (a) may comprise contacting a choroidal membrane with the lectin or contacting a Bruch's membrane with the lectin. Also useful are any other carbohydrate-binding agent, including non-protein molecules, that may be functional substitutes of lectins.

The lectin may be labeled, or may be unlabeled, wherein the method further comprises an additional step of contacting the choroidal membrane or Bruch's membrane with an agent that permits detection of bound lectin. The lectin may be selected from the group consisting of SBA, VVA, UEA-1 and sWGA. The method may further comprise making a treatment decision based on the distribution of lectin binding on the choroidal membrane and/or Bruch's membrane. Steps (a)-(c) may be performed a second time and the results from both identifications are compared.

In still yet another embodiment, there is provided a method of targeting a therapeutic agent to a choroidal neovascularization comprising (a) providing a lectin coupled to a therapeutic agent; and (b) administering the lectin to the eye of a subject in need thereof. Step (b) may comprise injection into the choroidal membrane, injection into the Bruch's membrane, injection into the subretinal lumen, topical application to the ocular sclera, or systemic administration. The lectin may be selected from the group consisting of SBA, VVA, UEA-1 and sWGA. The therapeutic agent may be Visudyne or an anti-angiongenic agent. Also useful are any other carbohydrate-binding agent, including non-protein molecules, that may be functional substitutes of lectins.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—Histopathology of case 1. (FIG. 1A) Area of disciform scarring reacts with Masson's Trichrome to give a blue staining pattern. Note the RPE pigment and the small vessel with red blood cells embedded in the scar (arrow). The outer nuclear layer shows significant degeneration. Scale bar=100 μm. (FIG. 1B) A subRPE neovascular membrane is present in this eye, with degeneration of the overlying retina. This membrane is primarily located between the dystrophic RPE (white arrows) and Bruch's membrane (black arrows). (H&E stain, scale bar=100 μm) (FIG. 1C) Higher magnification of FIG. 1B. (H&E stain, scale bar=50 μm).

FIGS. 2A-C—Histopathology of case 2 and case 3. (FIG. 2A) Low magnification view of case 2 showing cystic changes in the neural retina overlying the area of neovascularization. (H&E stain, collected with 2.5× objective lens). (FIG. 2B) PAS reactivity of BlamD in case 2 and the outer layers of Bruch's membrane (arrows). Several vascular elements are present in the space between these two matrices (asterisks), the outer nuclear layer is attenuated, and cystic changes are present within the inner nuclear layer (PAS stain, scale bar=100 μm). (FIG. 2C) In case 3, a CNVM is observed between the RPE and the outer layers of Bruch's membrane (H&E stain; asterisks: blood vessels; scale bar=50 μm).

FIGS. 3A-C—Matrix labeling with some lectins. (FIG. 3A) PNA labels a photoreceptor rosette (see (Rayborn et al., 1997) and the material within the scar (asterisk). (FIG. 3B) VVA reacts with basement membrane material in the subretinal space (arrows). (FIG. 3C) When utilized at higher concentrations, sWGA reacts with the layer of basal laminar deposit (asterisk) and choriocapillaris blood vessels. Lectin labeling: red fluorescence; DAPI: blue; RPE autofluorescence: orange-yellow. FIG. 3A: scale bar=100 μm; FIGS. 3B-C: scale bar=50 μm.

FIGS. 4A-C—Reaction of choroidal neovessels with the fucose-binding lectin UEA-I. All viable vessels in case 1 (FIG. 4A) and case 2 (FIG. 4B) were labeled with UEA-I (asterisks). (FIG. 4C) Very minor fluorescence of some vessels was observed with the avidin-Texas red reagent alone (Case 2). For FIGS. 4A-C, scale bars=50 μm. Lectin labeling: red fluorescence; DAPI: blue; RPE autofluorescence: orange-yellow.

FIGS. 5A-D—Lectin reactivity of vessels in choroidal neovascular membranes. (FIG. 5A) VVA labeling of cone inner segments and CNV vasculature (case 2). (FIG. 5B) A SBA-reactive vessel in a CNVM sends a branch (arrow) into the layer of BlamD (case 2). (FIG. 5C) A flat layer of vessels in case 1 is positive for SBA. (FIG. 5D) Vessels in case 1 reactive for sWGA (asterisk). Lectin labeling: red fluorescence; DAPI: blue; RPE autofluorescence: orange-yellow. Scale bars=50 μm.

FIGS. 6A-C—Histochemistry of a feeder vessel in a CNVM. Hematoxylin-eosin stain (FIG. 6A) of a large vessel breaching Bruch's membrane (at the asterisk). (FIG. 6B) Colocalization of collagen type IV (green) and SBA (red) in this vessel. Note the reactivity of SBA in the endothelium and surrounding matrix. (FIG. 6C) Colocalization of collagen type IV (green) and sWGA (red) in the same vessel shown in FIGS. 6A-B. Note that, unlike SBA, most of the sWGA binding is present on the endothelium. For FIGS. 6A-C, scale bars=50 μm.

### DESCRIPTION OF THE ILLUSTRATIVE EMBODIMENTS

### I. The Present Invention

Macular degeneration is the leading cause of blindness in individuals over 55. It is caused by the physical disturbance of the center of the retina, called the macula. The macula is the part of the retina which is responsible for the most acute and detailed vision. Therefore, it is critical for reading, driving, recognizing faces, watching television, and fine work. Even with a loss of central vision, however, color vision and peripheral vision may remain clear Vision loss usually occurs gradually and typically affects both eyes at different rates. The root causes of macular degeneration are still unknown.

There are two forms of age-related macular degeneration, “wet” and “dry.” Seventy percent of patients have the dry form, which involves thinning of the macular tissues and disturbances in its pigmentation. Thirty percent have the wet form, which can involve bleeding within and beneath the retina, opaque deposits, and eventually scar tissue. The wet form accounts for ninety percent of all cases of legal blindness in macular degeneration patients. Different forms of macular degeneration may occur in younger patients. These non-age related cases may be linked to heredity, diabetes, nutritional deficits, head injury, infection, contact lens abuse, or other factors.

In wet AMD, lesions call choroidal neovascularizations (CNV) are prevalent in choriodal and Bruch's membranes, which lead to subsequent disciform scarring (Gass, 1973) due to injury to the retinal pigment epithelium and photoreceptor cells of the neural retina. Over forty conditions have been associated with CNV (Green and Wilson, 1986). Of these age related macular degeneration (AMD) is the most prevalent. The vast majority of patients with AMD who suffer from legal blindness are affected with this neovascular form of the condition. Indeed, it is estimated that 90% of eyes legally blind due to AMD have CNV (Ferris et al., 1984), underscoring the fact that, although it affects only a fraction of all AMD patients, CNV is the major cause of vision loss in this disease. Unfortunately, the overall visual prognosis for CNV remains dismal. Severe visual loss occurs in over 60% of CNV cases over a five year period (Macular Photocoagulation Study Group, 1994a; 1994b; Macular Photocoagulation Study Group, 1991).

Structurally, CNVMs are comprised of both cellular and non-cellular elements. Cell types described as present within CNVMs include macrophages, RPE cells, and endothelial cells. The growth of blood vessels into the sub-RPE/subretinal space often creates a cleavage plane between Bruch's membrane and a detached layer of basal laminar deposit and/or the dystrophic RPE. Of primary interest in choroidal neovasculrization are the endothelial cells which may leak and/or hemorrhage, leading to disciform scarring of the macula. Whereas lectin binding patterns are observed on other structures within the CNVM, the lectin binding pattern to abnormal EC within the CNVM is potentially exploitable as a means of identifying and/or treating abnormal, as compared to normal, EC.

The inventor has performed a lectin histochemical assay of choroidal neovascular membranes (CNVMs) from three donors to determine whether a specific carbohydrate composition is associated with the neovascular complex. He found that a number of carbohydrate moieties were present on the vascular elements of CNVMs and disciform scars, including those recognized by SBA, VVA, UEA-I, and sWGA. SBA and sWGA were found to recognize the vascular elements of CNVMs at concentrations that failed to show strong labeling of normal vessels of the retina and choroid. Thus, it is proposed that these lectins can not only identify CNVs in situ and hence provide an early diagnosis of wet AMD, but they can also serve to target therapeutic agents to these lesions.

### II. Lectins

It has long been known that extracts from certain plants could agglutinate red blood cells. Although the term “lectin” was originally a term used to describe agglutinins which could discriminate among types of red blood cells. However, the term is used more defined as sugar-binding proteins from a wide variety of sources. Lectins have been found in plants, viruses, microorganisms and animals. Although lectins share the common property of binding to defined sugar structures, their roles in various organisms are not likely to be the same and remain incompletely understood.

Most lectins studied to date are multimeric, consisting of non-covalently associated subunits. It is this multimeric structure which gives lectins their ability to agglutinate cells or form precipitates with glycoconjugates in a manner similar to antigen-antibody interactions. Although most lectins can agglutinate some cell type, cellular agglutination is not a prerequisite. Some lectins can bind to cells and not cause agglutination, or the lectin may not bind to cells at all. The latter property may be a consequence of the structure of the lectin or the absence of a suitable receptor oligosaccharide on the cell surface.

Because of the specificity that each lectin has toward a particular carbohydrate structure, even oligosaccharides with identical sugar compositions can be distinguished or separated. Some lectins will bind only to structures with mannose or glucose residues, while others may recognize only galactose residues. Some lectins require that the particular sugar be in a terminal non-reducing position in the oligosaccharide, while others can bind to sugars within the oligosaccharide chain. Some lectins do not discriminate between a and b anomers, while others require not only the correct anomeric structure but a specific sequence of sugars for binding. The affinity between a lectin and its receptor may vary a great deal due to small changes in the carbohydrate structure of the receptor.

Lectin histochemistry is a morphological technique that takes advantage of the carbohydrate binding characteristics of various plant, animal and fungal proteins (Danguy et al., 1998). Different cell types, and cells under different environmental influences, alter their surface carbohydrate composition, and these alterations may be detected histologically or biochemically. This approach has been utilized on eyes with early AMD, to determine compositional characteristics of drusen and basal laminar deposits (Kliffen et al., 1994; Mulins et al., 1997; Mullins et al., 1999).

In the study discussed below, the inventor examined the carbohydrate moieties present in CNV and compared the labeling pattern of CNV with normal retinal and choroidal blood vessels. It was found that lectins able to identify CNV lesions and to distinguish normal vascularizations. This also presents the ability of lectins to selectively target therapeutic agents, including anti-angiogenic factors, to these lesions.

sWGA. Wheat germ agglutinin is derived from Triticum vulgaris (wheat germ). A succinylated derivative, sWGA, has been reported to have properties distinct from the native lectin. sWGA is an acidic protein with a pI of 4.0+/−0.2 while the native lectin is basic, pI of 8.5. The solubility of succinylated wheat germ agglutinin is about 100 times higher than that of the unmodified lectin at neutral pH. Both lectins are dimeric at pH down to 5, and the dissociation occurs at pH lower than 4.5. The binding of oligosaccharides of N-acetylglucosamine to both lectins is very similar on the basis of fluorescence and phosphorescence studies. The minimal concentration required to agglutinate rabbit red blood cells is about 2 microgram/ml with both lectins and the concentrations of N-acetylglucosamine and di-N-acetylchitobiose which inhibit agglutination are similar with both lectins (Monsigny et al., 1979).

The number of succinylated wheat germ agglutinin molecules bound to the surface of mouse thymocytes is ten times lower than that of the unmodified lectin although the apparent binding constant was only slightly different between the two lectins (Monsigny et al., 1979). Using conjugates of the native lectin and the succinylated form can provide a system to distinguish between sialylated glycoconjugates and those containing only N-acetylglucosamine structures.

SBA. Soybean agglutinin (SBA) is isolated from from Glycine max (soybean) seeds. Composed of four subunits of approximately equal size, soybean agglutinin is a family of closely related isolectins. This glycoprotein has a molecular weight of about 120,000 and an isoelectric point near pH 6.0. The monomeric species is found at pH 2.0 and below. The conformational stabilities of the tetramer and the monomer at the temperature of their maximum stabilities (310K) are 59.2 kcal/mol and 9.8 kcal/mol, respectively, indicating that oligomerization contributes significantly to the stability of the native molecule. Evidence suggests that the major hydrophobic core is present in the monomer itself and oligomerization involves mainly ionic interactions.

SBA preferentially binds to oligosaccharide structures with terminal α- or β-linked N-acetylgalactosamine, and to a lesser extent, galactose residues. Binding can be blocked by substitutions on penultimate sugars, such as fucose attached to the penultimate galactose in blood group B substance. SBA has been used in glycoprotein fractionation, histochemical applications and cell sorter analysis. An important application for SBA is the separation of pluripotential stem cells from human bone marrow.

### III. Assays for Lectin Binding

The present invention provides assays for the detection of CNV using lectins as selective binding agents. Generally, the carbohydrate composition of CNVMs will be exploited in order to detect the abnormal endothelial cells that reside in these membranes. Subjects will be treated through a variety of different routes—local, regional or systemic—with lectins or carbohydrate-binding molecules. These reagents may be labeled for direct detection (including the use of photo/laser interrogation), or they may be detected indirectly using a secondary agent (antibody or other hapten-binding reagent like biotin-avidin). Once CNVM is detected, the physician may make a diagnosis of wet AMD. In addition, the physician may make treatment decisions and effect therapies. The progress of these therapies, or simply the progression of the disease, may be monitored.

### IV. Linking Therapeutic and Diagnostic Agents to Lectins

A. Linking Technologies

In some embodiments, one may wish to link therapeutic or diagnostic reagents to lectins. A wide variety of coupling technologies may be used. For example, reagents may be used to directly attach agents to lectins. For example, photoaffinity agents such as iodinatable cross-linking agent N-hydroxysuccinimidyl-4-azidosalicylic acid (ASA) or sulfosuccinimidyl 2-(p-azidosalicylamido)ethyl-1,3′-dithiopropionate may be used.

Alternatively, linkers may be used to “bridge” between the lectin and the agent of choice. Such linkers may include a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

TABLE 1
HETERO-BIFUNCTIONAL CROSS-LINKERS

| | | | Spacer Arm | |
|---|---|---|---|---|
| | | | Length\after cross- | |
| linker | Reactive Toward | Advantages and Applications | linking | |
| SMPT | Primary amines | Greater stability | 11.2 | A |
| | Sulfhydryls | | | |
| SPDP | Primary amines | Thiolation | 6.8 | A |
| | Sulfhydryls | Cleavable cross-linking | | |
| LC-SPDP | Primary amines | Extended spacer arm | 15.6 | A |
| | Sulfhydryls | | | |
| Sulfo-LC-SPDP | Primary amines | Extended spacer arm | 15.6 | A |
| | Sulfhydryls | Water-soluble | | |
| SMCC | Primary amines | Stable maleimide reactive group | 11.6 | A |
| | Sulfhydryls | Enzyme-antibody conjugation | | |
| | | Hapten-carrier protein conjugation | | |
| Sulfo-SMCC | Primary amines | Stable maleimide reactive group | 11.6 | A |
| | Sulfhydryls | Water-soluble | | |
| | | Enzyme-antibody conjugation | | |
| MBS | Primary amines | Enzyme-antibody conjugation | 9.9 | A |
| | Sulfhydryls | Hapten-carrier protein conjugation | | |
| Sulfo-MBS | Primary amines | Water-soluble | 9.9 | A |
| | Sulfhydryls | | | |
| SIAB | Primary amines | Enzyme-antibody conjugation | 10.6 | A |
| | Sulfhydryls | | | |
| Sulfo-SIAB | Primary amines | Water-soluble | 10.6 | A |
| | Sulfhydryls | | | |
| SMPB | Primary amines | Extended spacer arm | 14.5 | A |
| | Sulfhydryls | Enzyme-antibody conjugation | | |
| Sulfo-SMPB | Primary amines | Extended spacer arm | 14.5 | A |
| | Sulfhydryls | Water-soluble | | |
| EDC/Sulfo-NHS | Primary amines | Hapten-Carrier conjugation | 0 | |
| | Carboxyl groups | | | |
| ABH | Carbohydrates | Reacts with sugar groups | 11.9 | A |
| | Nonselective | | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1988). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

B. Diagnostic Agents

A wide variety of diagnostic agents may be used in accordance with the present invention. For example, optical imaging with dyes permit visualization of biological activities (Blasdel et al., 1986; Grinvald et al., 1988; Kauer et al., 1988; Lieke et al., 1989). Dyes that are sensitive to physicochemical environments (such as pressure, cell membrane potential, ion concentration, acidity, partial pressure of oxygen, etc.), are subject to changes in absorption or emission of light. The resulting changes act as optical probes to transform biological activities into optical signals that can be converted into optical images.

Water soluble dyes are particularly well-suited, including acid dyes, basic dyes, direct dyes, and so on, and equivalents thereof. The dye composition may be prepared as a dry material for ease of storage and packaging. If prepared as a dry composition, prior to usage the composition may be prepared as a solution using a suitable liquid, including water and various organic solvents, or mixtures thereof and so on, by techniques well known to those skilled in the art.

Dyes include methylene blue, Tartrazine (CI 19140), Quinoline Yellow (CI 47005), Eosin (CI 45380), Acid Phloxine (CI 45410), Erythrosine (CI 45430), Sunset Yellow FCF (CI 15985), Acid Violet 5B (CI 42640), Patent Blue AF (CI 42080), Brilliant Cyanine 6B (CI 42660), Acid Brilliant Blue FCF (CI 42090), Naphthalene Green VSC (CI 44025) and Acid Blue Black 10B (CI 20470); and direct dyes such as Paper Yellow GG (CI Direct Yellow 131), Direct Scarlet 4BS (CI 29160), Congo Red (CI 22120), Violet BB (CI 27905), Direct Sky Blue 5B (CI 24400), Patent Blue Violet, Sulfan Dye), Pentamine, guajazulen blue Pentamine, Phthalocyanine Blue (CI 74180), Black G (CI 35255) and Deep Black XA (CI Direct Black 154). The CT number in the description above indicates the identification number in the Color Index, 3rd Ed., The Society of Dyers and Colorists, Bradford, Yorkshire (1971). Prefered dyes include Isosulfan blue or other dye which travels through the lymphatic system.

Chromophores include Fluorescein, Rhodamine, Acid Fuchsin; Acridine Orange; Acridine Red; Acridine Yellow; Alizarin Red; Allophycocyanin; Astrazon Brilliant; Astrazon Orange R; Astrazon Red 6B; Astrazon Yellow; Bodipy Fl; Bodipy TMR; Bodipy TR; Calcein; Calcein Blue; Calcium Green; Calcium Orange; Calcofluor White; Cascade Blue; Flazo Orange; Fluorescein Isothiocyanate (FITC); Fura-2; Fura Red; Genacryl Brilliant Red B; Genacryl Brilliant Yellow LOGF; Genacryl Pink 3G; Genacryl Yellow 5GF; Granular Blue; Lucifer Yellow CH; Lucifer Yellow VS; LysoSensor Blue DND-192, DND-167; LysoSensor Green DND-153, DND-189; LysoTracker Green; LysoTracker Yellow; LysoTracker Red; Magdala Red; Magnesium Green; Magnesium Orange; Mitotracker Green FM; Mitotracker Orange; Nile Red; Nuclear Fast Red; Nuclear Yellow; Oregon Green 488; Oregon Green 500; Oregon Green 514; Phorwite AR; Phorwite BKL; Phorwite Rev; Phorwite RPA; Pontochrome; Blue Black; Procion Yellow; Pyrozal Brilliant; Rhodamine Green; Rhodamine Red; Rhodol Green Fluorophore; Rose Bengal; Sevron Brilliant Red 2B; Sevron Brilliant Red 4G; Sevron Brilliant Red B; Sevron Orange; Sevron Yellow L; Texas Red; Thiozol Orange; True Blue; and Xylene Orange.

C. Therapeutic Agents

Visudyne Photodynamic therapy is an FDA-approved treatment for patients who have classic subfoveal choroidal neovascularization (CNV). Visudyne therapy is a two-step procedure that can be performed in a doctor's office. First, Visudyne, a light-sensitive drug (Verteporfin™ for injection), is injected intravenously into a patient's arm. Visudyne is taken up by the abnormal blood vessels in the eye. Second, the drug is activated by shining a non-thermal, or “cold” laser in the patient's eye. Visudyne therapy cannot restore vision lost to AMD, but it confines the retinal damage and slows the progression, of the disease.

Other agents that will find use in therapies against CNV include anti-angiogenics. These include Avastin, VEGF-Trap, NM-3, Neovastat, IMC-1C11, SU5416, SU6668, PTK787/ZK222584, SU11248, ZD6474, CP-547,632, Endostatin, Angiostatin, TNP-470, Thrombospondin-1, Vitaxin, Cilengitide, Combrestatin A4, ZD6126, 2-methoxyestradiol, DMXAA, Thalidomide, BMS-275291 and Celecoxib.

### V. Treatments for Choroidal Neovascularization

In general, the currently available therapeutic modalities are only able to decrease the extent to which vision is lost and are incapable of restoring vision (Bressler et al., 2001; Gragoudas et al., 2004; Ferris, 2004; Bressler, 2004). Thus, although no medical treatments have proven to be a cure for choroidal neovascularization, particular antiangiogenic substances such as thalidomide, angiostatic steroid, and metalloproteinase inhibitors are currently being tested. Through surgical testing, partial removal of choroidal neovascularization proved to be useless. Therefore the focus has been placed on photodynamic therapy, a procedure approved by the Food and Drug Administration.

In choroidal neovascularization patients, the fluid and blood along with the formation of new blood vessels form scar tissues which are trying to repair damages but are ultimately the cause of blindness. Photodynamic therapy is a treatment meant to stop the fluid as well as stunt further growth of the blood vessels among patients. Photodynamic therapy is performed in two phases. In the first phase, Visudyne (a special dye that only attaches itself to abnormal blood vessels underneath the retina) is injected. Then a laser which does not damage the retina activates a compound which closes the anomalous blood vessels located in the eye. CNV has been seen to disappear 24 hours after the procedure. Unfortunately, CNV has also been seen to reappear 2-3 months later in almost all the patients and long-term benefits are still unknown. However, in a year-long Treatment of Age-related Macular Degeneration study of 609 patients 16% of treated patients and 7% of placebo patients had visual improvement.

As discussed above, some scientists have suggested an association between macular degeneration and high saturated fat, low carotenoid pigments, and other substances in the diet. There is evidence that eating fresh fruits and dark green, leafy vegetables (such as spinach and collard greens) may delay or reduce the severity of age-related macular degeneration. Taking anti-oxidants like vitamins C and E may also have positive effects. Zinc, however, has shown mixed results. In some people, the long-term use of zinc causes digestive problems and anemia; its use is probably not worth the potential problems. Selenium is sometimes recommended.

Surgery to remove the scar produced by macular degeneration has been successful in younger patients, but less successful in older patients. If the degeneration is associated with leaking blood vessels in the center of the macula, and vision is worse than 20/70, laser surgery, called photocoagulation, is recommended. This will not improve vision but generally reduces further vision loss. Retinal transplantation is a new experimental approach to macular degeneration, but will require additional clinical research to determine safety and effectiveness. Another type of surgery is an experimental procedure known as submacular surgery. This procedure is performed from the inside of the eye in order to work on the retinal tissues to remove and replace the vitreous fluid. The downside of this procedure is that in order to heal the patient must be face-down for several weeks after the fluid is replaced.

Another factor is uv-radiation. It has been demonstrated that the blue rays of the spectrum seem to accelerate macular degeneration more than other rays of the spectrum. This means that very bright light, such as sunlight or its reflection in the ocean and desert, may worsen macular degeneration. Special sunglasses that block out the blue end of the spectrum may decrease the progress of the disease. Hypertension tends to make some forms of macular degeneration worse, especially in the wet form where the retinal tissues are invaded by new blood vessels. Finally, smoking or exposure to tobacco smoke can accelerate the development of the wet type of macular degeneration. Thus, mitigation of one or more of these risk factors also constitutes a useful treatment.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for Its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Aged donor eyes from 3 individuals with CNVMS were received from the Iowa Lions Eye Bank (Iowa City, Iowa). Eyecups or macular punches were either fixed (cases 1 and 2) or were embedded unfixed (case 3), as described below. Eyes were fixed by immersion for 2 hours in 4% paraformaldehyde solution diluted in 10 mM phosphate buffered saline, pH 7.4. Eyes were fixed within 5 hours of death. Maculae were washed in PBS and were then infiltrated and embedded in sucrose solution Barthel and Raymond, 1990). The maculae from these eyes were serially sectioned on a Microm HM505E cryostat and employed in the lectin histochemical study. Labeling patterns of the CNVMs in these eyes were compared with the patterns in normal retinal and choroidal endothelial cells (ECs) in these same eyes.

In order to determine whether the sucrose infiltration and embedment affected the pattern of labeling, a third eye with subRPE CNV (case 3) was embedded unfixed in optimal cutting temperature medium (OCT; Ted Pella; Redding, Calif.) within 12 hours of death, and frozen sections from this donor were evaluated with the same battery of lectins.

Patterns of lectin labeling in two eyes with choroidal neovascularization were evaluated. The panel of biotinylated lectins and their specificities are shown in Table 2. All lectins were obtained from Vector Laboratories (Burlingame, Calif.). Lectin labeling was performed essentially as described previously (Mullins et al., 1997) except that biotinylated lectins were utilized, followed by detection with avidin-Texas red (Vector Labs). The divalent cations MgCl₂ and CaCl₂ (1 mg/mL each) were included in all steps of the labeling protocol. Sections were blocked for 15 min in PBS with 1 mg/mL bovine serum albumin (Sigma, St. Louis, Mo.). Sections were then incubated in the biotinylated lectin diluted 1:50 to 1:400 in PBS (with albumin, MgCl₂ and CaCl₂) for 30 min, followed by 3×5 min washes, and incubation in avidin-Texas red (25 μg/mL) and the nuclear counterstain DAPI (4′-6-diamidino-2-phenylindole) for 30 min. Following 3×5 min washes in PBS, sections were coverslipped in Aquamount (Lerner Laboratories, Pittsburgh, Pa.).

Additional sections were stained using conventional hematoxylin/eosin staining (H&E), periodic acid-Schiff (PAS), or Masson's trichrome (MT). PAS and MT staining were performed at The University of Iowa F. C. Blodi Ocular Pathology Laboratory.

Sections were photographed on an Olympus BX41 microscope with a fluorescence attachment and filter sets for fluorescein, rhodamine, and DAPI. In order to discriminate between autofluorescence and Texas red labeling (particularly in areas of RPE lipofuscin, which is highly autofluorescent), all photographed fields were imaged in red, green and blue channels.

Eyes were also labeled with antibodies directed against type IV collagen (Chemicon rabbit polyclonal antibody) to visualize vascular basal laminae as well as NBT/BCIP (Vector Laboratories) in order to visualize the endogenous alkaline phosphatase activity of endothelial cells (McLeod and Lutty, 1994; Mullins et al., 2000). Immunohistochemical labeling was performed as described previously.

### Example 2

### Results

The three eyes with neovascular membranes and disciform scars showed features typical of those described in the literature for CNVMs associated with AMD (Small et al., 1976; Green et al., 1985; Bressler et al., 1992; Sarks et al., 1997; Grossniklaus et al., 2000) reviewed in Green (1999).

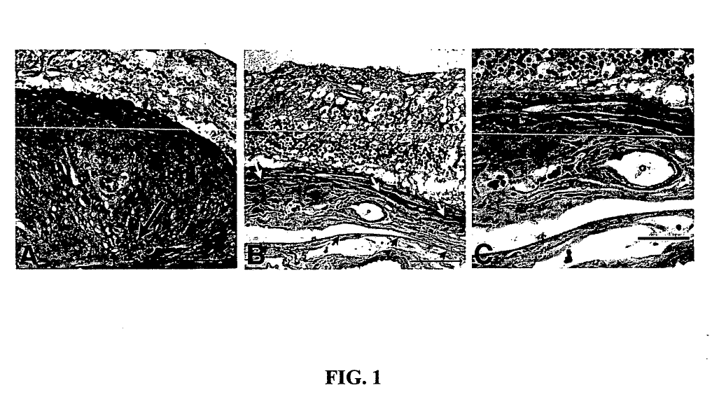

In case 1, a large, compact subfoveal scar was noted that was eosinophilic and stained blue on MT stain, suggesting a significant amount of collagen (FIG. 1A). The scar contained fibrovascular material that was present on both sides of a detached layer of basal laminar deposit with a discontinuous RPE layer on its inner surface. Islands of RPE were noted within the scar, as were rare vessels (FIG. 1A). Photoreceptor degeneration was noted above the scar, and several rosettes were present in the overlying retina. At the temporal edge of the scar, a flat layer of active blood vessels was noted lying between the outer aspect of Bruch's membrane and the degenerated RPE (FIGS. 1B-C).

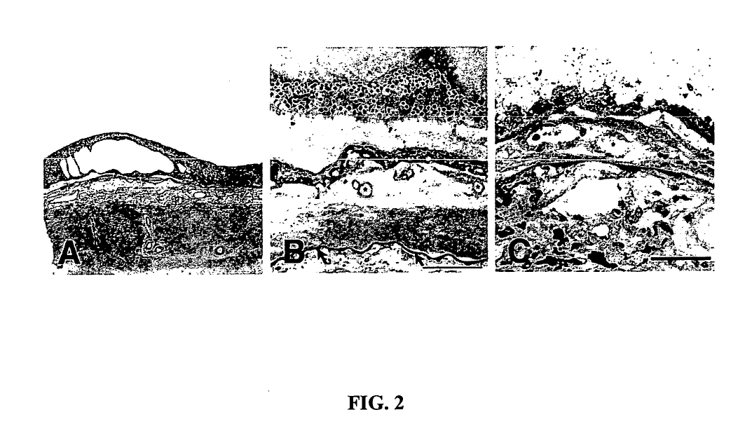

In case 2, a two-layered CNVM was present. The retina overlying the CNVM exhibited severe cystic changes at the level of the inner nuclear layer (FIG. 2A). In addition, the outer nuclear layer and photoreceptor outer segments were extremely attenuated. A detached layer of meandering basal laminar deposit (BlamD) was also noted, with a layer of loose proteinaceous material between the outer layers of Bruch's membrane and the layer of BlamD (Bressler et al., 1992) (FIG. 2B). PAS-reactive material in the BlamD and the residual Bruch's membrane was observed, with active blood vessels and matrix elements occupying this space (FIG. 2B). Case 3, an unfixed eye with a comparatively long death-preservation time, showed similar characteristics, except that the CNVM was located only in the subRPE space, between a layer of BlamD and Bruch's membrane (FIG. 2C).

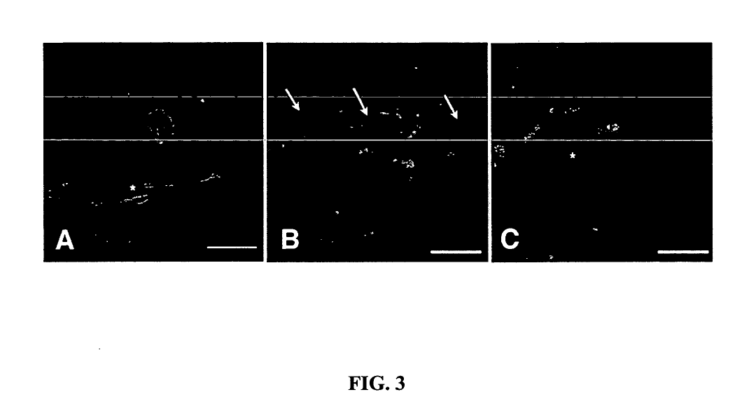

The labeling patterns of 11 lectins were determined in the retinal blood vessels, choroidal blood vessels, the glycoconjugates within the scar, and the vascular components of the neovascular membrane. The observed patterns are described in Table 2 and in FIGS. 3A-6C.

Several lectins showed intense labeling of matrix elements within the CNVM and/or disciform scar. The material within the scar was labeled with PNA (in case 2 only; FIG. 2A) and PSL (in cases 1 and 2). A sheet of matrix in the subretinal space, most likely corresponding to the basal lamina of dystrophic RPE cells, was labeled with SBA, PNA and VVA (FIG. 3B). When used at higher concentrations (20 μg/mL), sWGA labeled basal laminar deposits (FIG. 3C). At this concentration, sWGA also labeled normal retina and choroidal vasculature. PNA also labeled photoreceptor rosettes, which were observed in the overlying retina, as described previously (FIG. 3A) (Raybom et al., 1997).

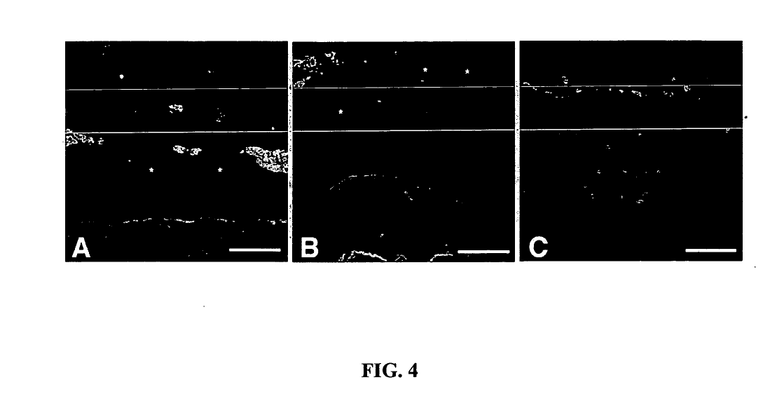

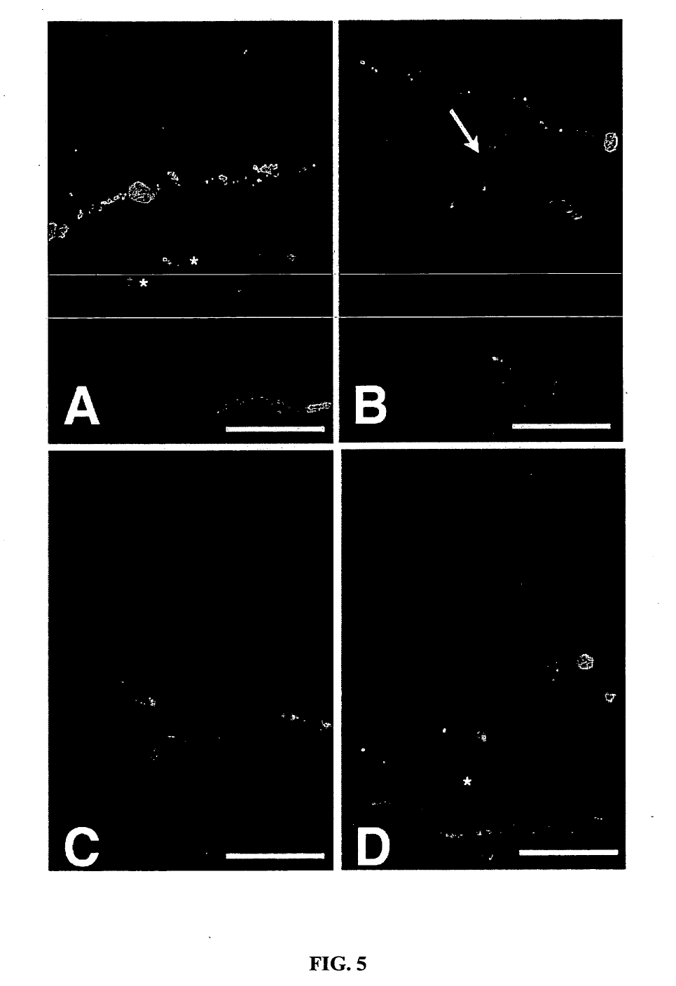

Vessels in the CNVM were labeled with UEA-I (which labeled all vessels in the eyes studied, FIG. 4A, 4B). Reactivity of the CNVM vessels was also noted with VVA (FIG. 5A), PSL, SBA (FIGS. 5B-C), and sWGA (FIG. 5D). These probes also showed variable labeling of vessels in normal retina and/or choroid. SBA was notably higher in CNV vessels in case 2 than in normal retinal and choroidal vessels, and exhibited positive reactivity of these vessels at relatively low concentrations (2.5 μg/mL) that did not label other structurcs in the eye. Similarly, sWGA labeled choroidal neovessels at concentrations at which other vascular beds were unlabeled or very weakly labeled. Similar results were obtained between sucrose embedded (cases 1 and 2) and unfixed (case 3) CNVMs (Table 2).

TABLE 2

| LECTIN | SPECIFICITY | CASE 1 | CASE 2 | CASE 3 |
|---|---|---|---|---|
| Canavalia ensiformis | alpha-linked mannose | BlamD+ | BlamD+ | [unable to assess] |
| (Con A) | | Scar + | Scar + | |
| | | CNV BV + | CNV BV + | |
| Dolichos bifloras (DBA) | alpha-linked Gal NAc | BlamD − | BlamD − | BlamD − |
| | | Scar − | Scar − | Scar − |
| | | CNV BV − | CNV BV +/− subset | CNV BV − |
| Arachis hypogea (PNA) | galactosyl (beta-1,3) GalNAc | BlamD − | BlamD − | BlamD − |
| | | Scar − | Scar ++ | Scar +/− |
| | | CNV BV − | CNV BV subset + | CNV BV −/+ |
| Glycine max (SBA) | alpha or beta linked GalNAc | BlamD − | BlamD − | BlamD − |
| | | Scar − | Scar − | Scar − |
| | | CNV BV + | CNV BV + | CNV BV + |
| Ulex europaeus I (UEA-I) | Alpha-linked fucose | BlamD − | BlamD − | BlamD − |
| | | Scar − | Scar − | Scar − |
| | | CNV BV + | CNV BV + | CNV BV + |
| | | Normal bv + | Normal bv+ | Normal bv+ |
| Pisum sativum (PSL) | oligosaccharides with | BlamD +/− to + | BlamD + | [unable to assess] |
| | alpha-linked mannose | Scar ++ | Scar ++ | |
| | | CNV BV +/− | CNV BV + | |
| Vicia villosa (VVA) | Alpha- or beta-linked | BlamD + | BlamD − | BlamD − |
| | terminal of GalNAc | Scar − | Scar −/+ | Scar − |
| | | CNV BV +/− | CNV BV + | CNV BV + |
| Phaseolus vulgaris (PHA-L) | Complex oligosaccharides | BlamD − | BlamD +/− | [unable to assess] |
| | | Scar − | Scar −/+ | |
| | | CNV BV +/− in scar | CNV BV + | |
| Triticum vulgaris (Succinylated) | GlcNAc | BlamD +/− (outer) | BlamD + | BlamD + |
| (sWGA) | | Scar − | Scar − | Scar − |
| | | CNV BV + subset | CNV BV ++ | CNV BV + |
| Erythrina cristagalli (ECL) | Galactose, esp. gal | BlamD + regionally | BlamD +/− | [unable to assess] |
| | (beta-1,4) GalNAc | Scar − | Scar + | |
| | | CNV BV + in scar | CNV BV + | |
| Sambucus nigra (EBL) | NeuNAc alpha2-6 Gal > | BlamD + | BlamD + | BlamD + |
| | NeuNAc alpha 2,3 Gal | Scar + | Scar − | Scar + |
| | | CNV BV + | CNV BV +/− | High background |
| | | (<normal bv) | (<normal bv) | |

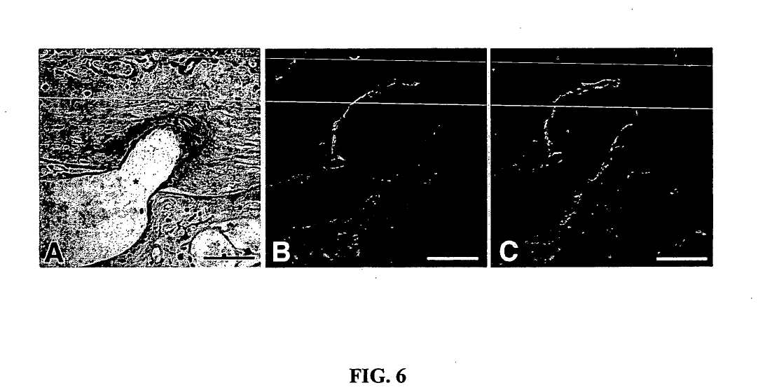

The inventors also sought to evaluate the glycoconjugates associated with a large feeder vessel breaching Bruch's membrane in case 2 (FIG. 6A). Two lectins found to react with subRPE vessels in CNV were utilized in dual labeling experiments with an antibody directed against type IV collagen. Both SBA (FIG. 6B) and sWGA (FIG. 6C) reacted with components of this large vessel. The SBA-reactive glycoconjugates appeared to be external to the inner layer of ECs, whereas sWGA appeared to localize to the EC surface internal to the layer of collagen IV (FIG. 6C).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference:
U.S. Pat. No. 4,680,338
U.S. Pat. No. 5,141,648
U.S. Pat. No. 5,563,250
U.S. Pat. No. 5,880,270
Attebo et al., Ophthalmology, 103:357-364, 1996.
Barthel and Raymond, J. Histochem. Cytochem., 38:1383-1388, 1990.
Blasdel and Salama, Nature, 321:579, 1986.
Bressler et al., Arch. Ophthalmol., 110(6):827-832, 1992.
Bressler et al., Arch. Ophthalmol., 119:198-207, 2001.
Bressler, JAMA., 291:1900-19001, 2004.
Danguy et al., Acta Anat. (Basel), 161:206-218, 1998.
Ferris et al., Arch. Ophthalmol., 102(11):1640-1642, 1984.
Ferris, N. Engl. J. Med., 351:2863-2865, 2004.
Friedman et al., Arch. Ophthalmol., 122(4):564-572, 2004.
Gass, Archives of Ophthal., 90:206-217, 1973.
Gragoudas et al., N. Engl. J. Med., 351:2805-2816, 2004.
Green and Wilson, Ophthalmology, 93(9):1169-1176, 1986.
Green et al., Ophthalmology, 92:615-627, 1985.
Green, Mol. Vis., 5:27, 1999.
Grinvald et al., Physiological Reviews, 68:1285, 1988.
Grossniklaus et al., Arch. Ophthalmol., 118:625-629, 2000.
Heiba et al., Genet. Epidemiol., 11:51-67, 1994.
Kauer, J. S. Nature, 331:166, 1988.
Klayer et al., Arch. Ophthalmol., 116:1646-1651, 1998.
Klayer et al., Arch. Ophthalmol., 116:653-658, 1998.
Klein et al., Ophthalmology, 99:933-943, 1992.
Kliffen et al., Invest. Ophthalmol. Visual Sci., 35:2901-2905, 1994.
Lieke et al., Annu. Rev. Physiol. 51:543, 1989.
Macular Photocoagulation Study Group, Arch. Ophthalmol., 109:1109-1114, 1991.
Macular Photocoagulation Study Group, Arch. Ophthalmol., 112:480-488, 1994.
Macular Photocoagulation Study Group, Arch. Ophthalmol., 112:500-509, 1994.
McLeod and Lutty, Invest. Ophthal. Visual Sci., 35:3799-3811, 1994.
Monsigny et al., Eur. J. Biochem. 98, 39 (1979).
Mullins et al., J. FASEB, 14:835-846, 2000.
Mullins et al., J. Histochem. Cytochem., 47:1533-1539, 1999.
Mullins et al., Ophthalmology, 104:288-294, 1997.
Rayborn et al., In: Degenerative Retinal Diseases, LaVail (Ed.), Plenum Press, 17-21, NY, 1997.
Sarks et al., Eye, 11:515-522, 1997.
Seddon et al., Am. J. Ophthalmol., 123:199-206, 1997.
Seddon et al., Jama, 272:1413-1420, 1994.
Small et al., Arch. Ophthalmol., 94:601-607, 1976.
Smith et al., Ophthalmology, 108:697-704, 2001.
Tielsch et al., N. Engl. J. Med., 332:1205-1209, 1995.
Wawrzynczak & Thorpe, Cancer Treat. Res., 37:239-251, 1988.

What is claimed is:

1. A method of identifying choroidal neovascularization (CNV) in a subject comprising:
(a) contacting a choroidal membrane and or a Bruch's membrane of said subject with a lectin;
(b) assessing the binding of said lectin to said choroidal membrane and/or Bruch's membrane; and
(c) comparing binding patterns of said lectins to the known structure of CNV.

2. The method of claim 1, wherein step (a) comprises contacting a choroidal membrane with said lectin.

3. The method of claim 1, wherein step (a) comprises contacting a Bruch's membrane with said lectin.

4. The method of claim 1, wherein said lectin is labeled.

5. The method of claim 1, wherein said lectin is unlabeled, and said method comprises an additional step of contacting said choroidal membrane or Bruch's membrane with an agent that permits detection of bound lectin.

6. The method of claim 1, wherein said lectin is selected from the group consisting of SBA, VVA, UEA-1 and sWGA.

7. The method of claim 6, wherein said lectin is SBA.

8. The method of claim 6, wherein said lectin is sWGA.

9. The method of claim 1, further comprising making a treatment decision based on the distribution of lectin binding on said choroidal membrane and/or Bruch's membrane.

10. The method of claim 1, wherein steps (a)-(c) are performed a second time and the results from both identifications are compared.

11. A method of diagnosing wet macular degeneration comprising:
(a) contacting a choroidal membrane and or a Bruch's membrane of a subject with a lectin;
(b) assessing the binding of said lectin to said choroidal membrane and/or Bruch's membrane; and
(c) comparing binding patterns of said lectins to the known structure of choroidal neovascularlization (CNV),
wherein the identification of a CNV structure in said choroidal membrane or Bruch's membrane is diagnostic of wet macular degeneration.

12. The method of claim 11, wherein step (a) comprises contacting a choroidal membrane with said lectin.

13. The method of claim 11, wherein step (a) comprises contacting a Bruch's membrane with said lectin.

14. The method of claim 11, wherein said lectin is labeled.

15. The method of claim 11, wherein said lectin is unlabeled, and said method comprises an additional step of contacting said choroidal membrane or Bruch's membrane with an agent that permits detection of bound lectin.

16. The method of claim 11, wherein said lectin is selected from the group consisting of SBA, VVA, UEA-1 and sWGA.

17. The method of claim 16, wherein said lectin is SBA.

18. The method of claim 16, wherein said lectin is sWGA.

19. The method of claim 11, further comprising making a treatment decision based on the distribution of lectin binding on said choroidal membrane and/or Bruch's membrane.

20. The method of claim 11, wherein steps (a)-(c) are performed a second time and the results from both identifications are compared.

31. A method of targeting a therapeutic agent to a choroidal neovascularization comprising:
(a) providing a lectin coupled to a therapeutic agent; and
(b) administering said lectin to the eye of a subject in need thereof.

32. The method of claim 31, wherein step (b) comprises injection into the choroidal membrane.

33. The method of claim 31, wherein step (b) comprises injection into the Bruch's membrane.

34. The method of claim 31, wherein step (b) comprises injection into the subretinal lumen.

35. The method of claim 31, wherein step (b) comprises topical application to the ocular sclera.

36. The method of claim 31, wherein step (b) comprises systemic administration.

37. The method of claim 31, wherein said lectin is selected from the group consisting of SBA, VVA, UEA-1 and sWGA.

38. The method of claim 36, wherein said lectin is SBA.

39. The method of claim 36, wherein said lectin is sWGA.

40. The method of claim 31, wherein the therapeutic agent is Visudyne.

41. The method of claim 31, wherein the therapeutic agent is an anti-angiongenic agent.
